(19) United States
(12) Patent Application Publication
(10) Pub. No.: US 2013/0096511 A1
(43) Pub. Date: Apr. 18, 2013

(54) TACTILE IDENTIFICATION OF DRUG FILLED CARTRIDGE

(71) Applicant: Ross Douglas Laurie MacArthur, Sandbach (GB)

(72) Inventor: Ross Douglas Laurie MacArthur, Sandbach (GB)

(73) Assignee: SANOFI-AVENTIS DEUTSCHLAND GMBH, Frankfurt am Main (DE)

(21) Appl. No.: 13/695,166

(22) PCT Filed: May 3, 2011
(86) PCT No.: PCT/EP2011/057039

§ 371 Date: Dec. 27, 2012

Related U.S. Application Data
(60) Provisional application No. 61/331,096, filed on May 4, 2010.

(30) Foreign Application Priority Data
Jul. 29, 2010 (EP) 10171171.1

(51) Int. Cl. A61J 1/18; A61M 5/31

(52) U.S. Cl. CPC A61J 1/18; A61M 5/3129
USPC 604/189; 604/404

(57) ABSTRACT

A tactile identification coding for a cartridge for use with a drug delivery device is described. The tactile identification coding includes the cartridge containing a drug and an identification coding located on the cartridge, the identification coding including a logical symbol to visually indicate to a user the type of drug contained in the cartridge. A cartridge holder may also be included to receive the cartridge, the cartridge holder having a second tactile identification coding that corresponds to the tactile identification coding on the cartridge.

### CROSS REFERENCE TO RELATED APPLICATIONS

The present application is a U.S. National Phase Application pursuant to 35 U.S.C. §371 of International Application No. PCT/EP2011/057039 filed May 3, 2011, which claims priority to U.S. Provisional Patent Application No. 61/331,096 filed May 4, 2010 and European Patent Application No. 10171171.1 filed Jul. 29, 2010. The entire disclosure contents of these applications are herewith incorporated by reference into the present application.

### FIELD OF INVENTION

The present patent application may be generally directed to reservoirs, particularly reservoirs containing a medicament. More particularly, the present application may be generally directed to a coding for identifying a type of drug for use with a reservoir and reservoir housing so as to prevent unwanted reservoir cross use. As just one example, such medicament reservoirs may comprise an ampoule, a cartridge assembly, a vial, or a pouch, and may be used with a medical delivery device. Exemplary medical delivery devices include, but are not limited to syringes, pen type syringes, pumps, inhalers, or other similar injection or infusing devices that require at least one reservoir containing at least one medicament.

### BACKGROUND

Medicament reservoirs such as ampoules, cartridge assemblies, or vials are generally known. Such reservoirs are especially used for medicaments that may be self administered by a patient. For example, with respect to insulin, a patient suffering from diabetes may require a certain amount of insulin to either be injected via a pen type injection syringe or infused via a pump. With respect to certain known reusable pen type drug delivery devices, a patient loads a cartridge containing the insulin into a proximal end of a cartridge housing. After the cartridge assembly has been correctly loaded, the user may then be called upon to select a dose of medicament. Multiple doses may be dosed from the cartridge assembly. Where the drug delivery device comprises a reusable device, once the cartridge assembly is empty, the cartridge housing is disconnected from the drug delivery device and the empty cartridge is removed and replaced with a new cartridge. Most suppliers of such cartridges recommend that the user dispose of the empty cartridges properly. Where the drug delivery device comprises a disposable device, once the cartridge assembly is empty, the user is recommended to dispose of the entire device.

Such known self administration systems requiring the removal and reloading of empty cartridges have certain limitations. For example, in certain generally known systems, a user simply loads a new cartridge assembly into the delivery system without the drug delivery device or without the cartridge having any mechanism of preventing cross use of an incorrect cartridge. That is, the drug delivery device does not have a mechanism for determining if the medicament contained in the cartridge is indeed the correct type of medicament to be administered by the patient. Alternatively, certain known drug delivery devices do not present a mechanism for determining if the correct type of medicament within the cartridge should be used with that particular drug delivery system. This potential problem could be exacerbated given that certain elderly patients, such as those suffering from diabetes, may have limited manual dexterity. Identifying an incorrect medicament is quite important, since the administration of a potentially incorrect dose of a medicament such as a short acting insulin in lieu of a long acting insulin could result in injury or even death.

Another concern that may arise with such disposable cartridges is that these cartridges are manufactured in essentially standard sizes and manufactured to comply with certain recognized local and international standards. Consequently, such cartridges are typically supplied in standard sized cartridges (e.g., 3 ml cartridges). Therefore, there may be a variety of cartridges supplied by a number of different suppliers and containing a different medicament but they may fit a single drug delivery device. As just one example, a first cartridge containing a first medicament from a first supplier may fit a medical delivery device provided by a second supplier. As such, a user might be able to load and then dispense an incorrect medicament (such as a rapid or basal type of insulin) into a drug delivery device without being aware that the medical delivery device was perhaps neither designed nor intended to be used with such a cartridge. As such, there is a growing desire from users, health care providers, care givers, regulatory entities, and medical device suppliers to reduce the potential risk of a user loading an incorrect drug type into a drug delivery device. There is also, therefore, a desire to reduce the risk of dispensing an incorrect medicament (or the wrong concentration of the medicament) from such a drug delivery device.

There is, therefore, a general need to physically dedicate or mechanically code a cartridge to its drug type and design an injection device that only accepts or works with the dedication or coded features provided on or with the cartridge so as to prevent unwanted cartridge cross use. Similarly, there is also a general need for a dedicated cartridge that allows the medical delivery device to be used with only an authorized cartridge containing a specific medicament while also preventing undesired cartridge cross use.

There is also a general need to provide a dedicated cartridge that is difficult to tamper with so that the cartridge may not be compromised in that the cartridge can be used with an unauthorized drug or drug delivery device. Because such cartridges may be difficult to tamper with, they may also reduce the risk of counterfeiting: i.e., making it more difficult for counterfeiters to provide unregulated counterfeit medicament carrying products.

It is an object of the present disclosure to facilitate provision of a drug delivery device with increased user safety.

This object is achieved, for example, by the subject matter of the independent claims. Advantageous embodiments and refinements are subject matter of the dependent claims.

### SUMMARY

According to an exemplary arrangement, a tactile identification coding for use with a drug cartridge is provided. The tactile identification coding includes a cartridge containing a drug and an identification coding located on the cartridge, the identification coding including a, preferably logical, symbol to visually and/or tactilely indicate to a user the type of drug contained in the cartridge.

In another arrangement, a drug delivery device is provided. The drug delivery device has a cartridge having an identification coding including a, preferably logical, symbol to visually and/or tactilely indicate to a user the type of drug contained in the cartridge. The drug delivery device furthermore has a cartridge holder to receive the cartridge. The cartridge may be retained in the cartridge holder. The cartridge holder includes a second tactile and/or visual identification coding that preferably corresponds to the, e.g. tactile, identification coding on the cartridge.

One aspect relates to a tactile identification system, comprising a cartridge containing a drug and an identification coding located on the cartridge. The, preferably tactile, identification coding may include a symbol, preferably a logical symbol, to visually and/or tactilely indicate to a user information about the drug, e.g. to indicate to the user the type of drug, contained in the cartridge.

A user may get information about the drug contained in the cartridge via the symbol. The user may gain the information tactilely and/or visually. Thus, the identification coding increases user safety as it provides information to the user about the drug. The symbol may be easily interpreted by the user, preferably tactilely and/or visually. The symbol is expediently visible from outside of the cartridge.

Preferably, the symbol indicates a drug action rate information, the drug action rate information including one of at least two different drug action rates. At least, the drug action rate information includes the information whether the drug action rate is “slow” or “fast”.

It is preferred that the drug action rate information is provided by the outer shape of the symbol. In this way, the drug action rate information may be derived directly by the user without any need for analyzing different aspects of the symbol. Preferably, the drug action rate information is provided by the outer shape alone.

Preferably, the symbol represents an animal. This enables the user to quickly read the code by recognizing the type of animal. Here, no detailed analysis of the outer shape is required, since animals are widely known by the users and as such the symbol will quickly be related an accordingly known representation of the animal.

Furthermore, it is preferred when the type of animal is typical for a drug action rate, i.e. at least typical for a fast or a slow drug action rate. In this case, the user may recognize the drug action rate without the need for the step of decoding a code since the representation of the code leads directly to the information that has to be indicated. This may enhance the comfort of the user when operating the device.

The symbol, particularly its shape, may be intuitively indicative for a characteristic of the drug, e.g. whether the cartridge contains a slow acting drug or a fast acting drug such as slow acting or fast acting insulin. Pictograms or other representations, e.g. pictograms or representations of animals, may be used as symbols for this purpose. If the drug is a fast acting drug, an animal which is known to move at high speed may be used. If the drug is a slow acting drug, an animal which is known to move at low speed may be used. For example, the identification coding, particularly the symbol, may comprise a turtle to indicate that the cartridge contains slow acting insulin or a hare to indicate that the cartridge contains fast acting insulin. The identification coding, in particular the symbol, may comprise a color. Preferably, the identification coding is distinguished and/or highlighted by the color from its surroundings.

The tactile identification system or coding may further comprise a cartridge holder, which is preferably configured to receive the cartridge or in which the cartridge is received. The cartridge holder may include or comprise a second tactile and/or visual identification coding. The second identification coding may correspond, e.g. in color, in shape and/or in the information it transports to the user, to the identification coding on the cartridge. The second identification coding may comprise a color. The identification coding and the second identification coding may comprise the same color. The identification coding may comprise a green turtle and the second identification coding may comprise the color green to indicate that the cartridge contains slow acting insulin, or the identification coding may comprise a pink hare and the second identification coding may comprise the color pink to indicate that the cartridge contains fast acting insulin.

The identification coding may comprise only one coding feature, e.g. the symbol, or a plurality of coding features, e.g. the symbol and one or more other features.

The identification coding may be secured to the cartridge by a fastener. The identification coding may be located on an outer surface of the cartridge.

The cartridge may be intended to be used with a drug delivery device. The device may be a reusable device or a disposable device.

A drug delivery device preferably comprises the tactile identification system or coding as described above. The drug delivery device may comprise a reusable drug delivery device or a disposable drug delivery device. Expediently, a cartridge holder is provided in the device to retain the cartridge.

In the following text, a set of further aspects of this disclosure is described. The aspects are denoted by numbers, which facilitates referencing the features contained in specific aspects by the respective number. These aspects are:

1. A tactile identification coding for use with a drug cartridge comprising:

a cartridge containing a drug;

an identification coding located on the cartridge, the identification coding including a logical symbol to visually indicate to a user the type of drug contained in the cartridge.

2. The tactile identification coding of aspect 1 wherein the identification coding comprises a turtle to indicate that a cartridge contains slow acting insulin.

3. The tactile identification coding of aspect 1 wherein the identification coding comprises a hare to indicate that a cartridge contains fast acting insulin.

4. The tactile identification coding of aspect 1 further comprising a cartridge holder configured to receive the cartridge.

5. The tactile identification coding of aspect 4 wherein the cartridge holder includes a second tactile identification coding that corresponds to the tactile identification coding on the cartridge.

6. The tactile identification coding of aspect 5 wherein the second tactile identification coding comprises a color.

7. The tactile identification coding of aspect 5 wherein the identification coding comprises a green turtle and the second tactile identification coding comprises the color green to indicate that a cartridge contains slow acting insulin.

8. The tactile identification coding of aspect 5 wherein the identification coding comprises a pink hare and the second tactile identification coding comprises the color pink to indicate that a cartridge contains fast acting insulin.

9. The tactile identification coding of aspect 1 wherein the identification coding comprises only one coding feature.

10. The tactile identification coding of aspect 1 wherein the identification coding comprises a plurality of coding features.

11. The tactile identification coding of aspect 1 wherein the identification coding is secured to the cartridge by a fastener.

12. The tactile identification coding of aspect 1 wherein the identification coding is located on an outer surface of the cartridge.

13. The tactile identification coding of aspect 1 wherein the drug cartridge is intended for use with a drug delivery device.

14. The tactile identification coding of aspect 13 wherein the drug delivery device comprises a reusable drug delivery device.

15. A drug delivery device comprising:

a cartridge having an identification coding including a logical symbol to visually indicate to a user the type of drug contained in the cartridge;

a cartridge holder to receive the cartridge, the cartridge holder including a second tactile identification coding that corresponds to the tactile identification coding on the cartridge.

16. The drug delivery device of aspect 15 wherein the identification coding comprises a turtle to indicate that a cartridge contains slow acting insulin.

17. The drug delivery device of aspect 15 wherein the identification coding comprises a hare to indicate that a cartridge contains fast acting insulin.

18. The drug delivery device of aspect 15 wherein the second tactile identification coding comprises a color.

19. The drug delivery device of aspect 18 wherein the identification coding comprises a green turtle and the second tactile identification coding comprises the color green to indicate that a cartridge contains slow acting insulin.

20. The drug delivery device of aspect 18 wherein the identification coding comprises a pink hare and the second tactile identification coding comprises the color pink to indicate that a cartridge contains fast acting insulin.

21. The drug delivery device of aspect 15 wherein the identification coding is secured to the cartridge by a fastener.

22. The drug delivery device of aspect 15 wherein the identification coding is located on an outer surface of the cartridge.

23. The drug delivery device of aspect 15 wherein the drug delivery device comprises a reusable drug delivery device

24. The drug delivery device of aspect 15 wherein the drug delivery device comprises a disposable drug delivery device.

Features, which are described above and below in connection with different aspects, arrangements or embodiments, may be combined with each other even if the combination is not explicitly described herein.

These as well as other features and advantages of various aspects of the present disclosure will become apparent to those of ordinary skill in the art by reading the following detailed description, with appropriate reference to the accompanying drawings.

### BRIEF DESCRIPTION OF THE DRAWINGS

Exemplary embodiments are described herein with reference to the drawings, in which:

FIG. 1a illustrates an exemplary pen type drug delivery device;

FIG. 1b illustrates an exemplary drug cartridge;

FIG. 2 illustrates a cartridge having a tactile identification coding;

FIG. 3 illustrates a cartridge and cartridge holder each having a tactile identification coding;

FIG. 4 illustrates another embodiment of a cartridge having tactile identification coding; and

FIG. 5 illustrates another embodiment of a cartridge and a cartridge holder each having a tactile identification coding.

### DETAILED DESCRIPTION

For purposes of the present application, a tactile identification coding is provided on a cartridge to provide a tactile and visual indication of the type of drug contained in the cartridge. The color or symbol used as the coding provides a logical and intuitive explanation to a user as to the type of drug. Thus, this coding ensures that the appropriate cartridge and drug can be correctly identified by the patient.

Such a cartridge may be used with any type of drug delivery device that utilizes a reservoir or cartridge, such as, for example, a reusable pen type drug delivery device, a disposable pen type delivery device, a prefilled syringe or any auto-injector.

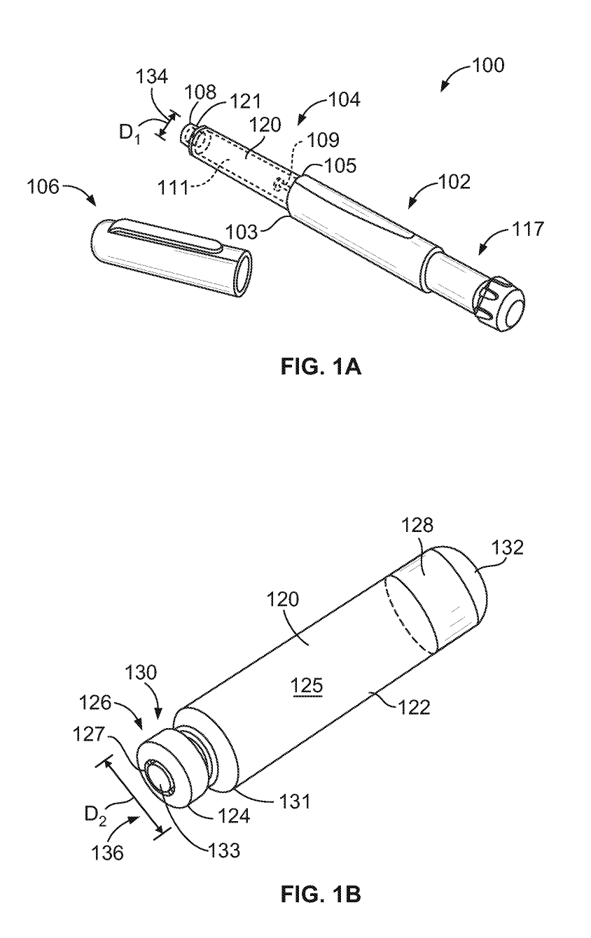

Referring to FIG. 1a, there is shown a drug delivery device 100 in the form of a pen type syringe. The drug delivery device 100 comprises a dose setting mechanism 102, a cartridge holder 104, and a removable cap 106. A proximal end 105 of the cartridge holder 104 and a distal end 103 of the dose setting mechanism 102 are removably secured together. The pen type syringe may comprise a re-usable or a disposable pen type syringe. Where the syringe comprises a re-usable device, the cartridge holder 104 and the dose setting mechanism are removably coupled together. In a disposable device, they are permanently coupled together. In FIG. 1, the dose setting mechanism 102 comprises a piston rod 109, such as a threaded piston rod that rotates when a dose is injected.

To inject a previously set dose, a double ended needle assembly (not shown) is attached to a distal end 108 of the cartridge holder. Preferably, the distal end of the holder comprises a thread 121 (or other suitable connecting mechanism such as a snap lock, snap fit, form fit, or bayonet lock mechanism) so that the needle assembly may be removably attached to the distal end of the holder. When the drug delivery device is not in use, the removable cap 106 can be releasably retained over the cartridge holder 104.

An inner cartridge cavity 111 defined by the cartridge holder 104 is dimensioned and configured to securely receive and retain a cartridge, such as glass cartridge 120. FIG. 1b illustrates a perspective view of the cartridge 120 that may be used with the drug delivery device illustrated in FIG. 1a. Typically, the cartridge 120 is manufactured of glass and includes a generally tubular barrel 122 extending from a distal end 130 to a proximal end 132. The cartridge 120 may be inserted into an inner bore 101 of the drug delivery device 100.

At the distal end 130, the cartridge 120 includes a smaller diameter neck 126 and this neck projects distally from the shoulder 131 of the barrel 122. Preferably, the smaller diameter neck 126 is provided with a large diameter annular bead 124 which extends circumferentially thereabout at the extreme distal end of the neck 126 and defines an opening 127. A pierceable seal or septum 133 is securely held across the opening 127 by a metallic sleeve or a ferrule.

Medicament 125 is pre-filled into the cartridge 120 and is retained within this cartridge, in part, by the pierceable seal 133, a ferrule, and a stopper 128. The stopper 128 is in sliding fluid-tight engagement with the inner tubular wall of the barrel 122. Axially directed forces acting upon the stopper 128 during dose injection or dose administration urge the medication 125 from the cartridge 120 though a double ended needle mounted onto the distal end 130 of the cartridge holder 104 and into the injection site. Such axially forces may be provided by the piston rod 109 working in unison with the dose setting member 102.

A portion of the cartridge holder 104 defining the cartridge holder cavity 111 is of substantially uniform diameter represented in FIG. 1a by D₁ 134. This diameter D₁ 134 is preferably slightly greater than the diameter D₂ 136 of the cartridge 120. The interior of the cartridge holder includes an inwardly-extending annular portion or stop that is dimensioned to prevent the cartridge 120 from moving within the cartridge holder 104. In this manner, when the cartridge 120 is loaded into the cavity 111 of the cartridge holder 104 and the cartridge holder 104 is then connected to the dose setting member 102, the cartridge assembly 120 will be securely held within the cartridge holder cavity 111. The cartridge holder 104 may also include a fastening mechanism for securing the cartridge holder 104 within the drug delivery device 100, which is described in more detail below.

A number of doses of a medicament 125 may be dispensed from the cartridge 120. Preferably, the cartridge 120 contains a type of medicament that must be administered often, such as one or more times a day. One such medicament is insulin.

The dose setting mechanism 102 comprises a dose setter 117 at the proximal end of the dose setting mechanism 102. In one preferred arrangement, the dose setter 117 is rotated to set a dose. To administer this set dose, the user attaches the needle assembly comprising a double ended needle on the distal end of the cartridge holder. In this manner, the needle assembly pierces the seal 133 of the cartridge 120 and is therefore in liquid communication with the medicament 125. The user pushes on the dose setter 117 to inject the set dose. The same dose setting and dose administration procedure is followed until the medicament 125 in the cartridge is expended and then a new cartridge must be loaded in the drug delivery device. To exchange an empty cartridge, the user is called upon to remove the cartridge holder 104 from the dose setting mechanism 102.

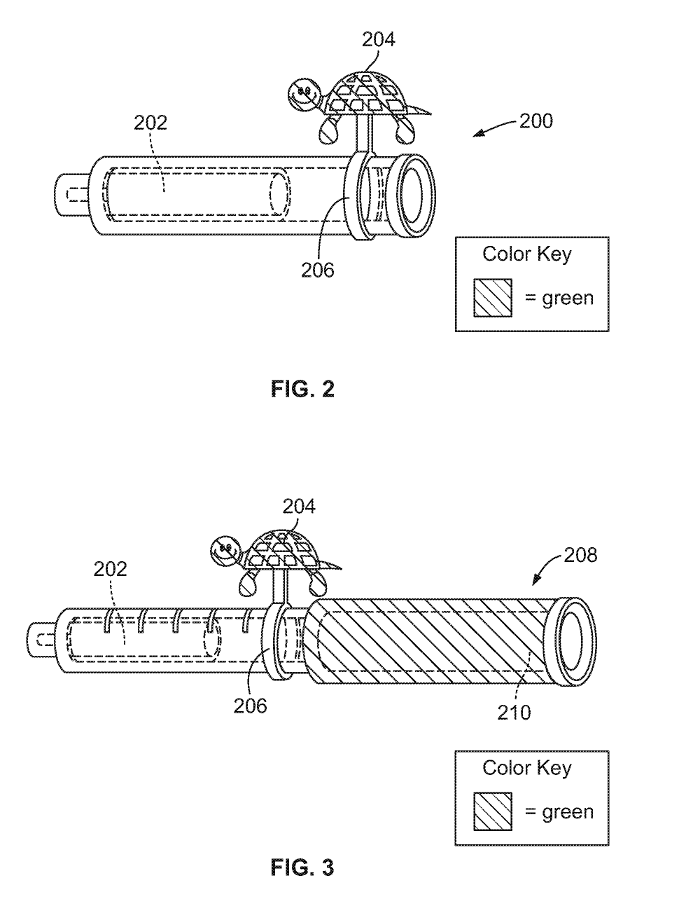

In accordance with exemplary embodiments, a cartridge, such as cartridge 120, or cartridge holder, such as cartridge holder 104, may be coded to a delivery device, so that given cartridges and cartridge holders may only be connected with intended drug delivery devices and vice versa. FIG. 2 illustrates a first arrangement of a cartridge 200 including a visual and/or tactile identification coding, such as coding 204. This coded cartridge may be connected to a drug delivery device, such as drug delivery device 100. The cartridge may also be attached to a cartridge holder that has a similarly coded portion. This similarly coded portion may be, for example, a color that matches the tactile coding 204. The coded cartridge 200 is intended for use with a drug delivery device similar to the drug delivery device of FIG. 1a, but a preferred drug delivery device for use with the coded cartridge 200 may have a slightly modified inner cavity. In addition, the removable cap 106 could also be modified to as to fit over the distal end of the cartridge 200.

FIG. 2 illustrates a first arrangement of a coded cartridge 200 for use with the drug delivery device 100. In this arrangement, the tactile identification coding 204 on the cartridge indicates a particular drug or medicament is contained in the cartridge 120. The cartridge may be inserted into a cartridge holder, such as cartridge holder 104, which is then inserted into the drug delivery device 100. The tactile identification coding 204 includes a logical, intuitive symbol to visually indicate to a user the type of drug contained in the cartridge. Thus, this system ensures that the appropriate cartridge and drug can be correctly identified by the patient.

The coded cartridge 200 may include a visual and tactile identification coding 204. The coding includes a logical symbol to visually indicate to a user the type of drug contained in the cartridge. For example, as shown in FIG. 2, the coding 204 includes a turtle, which is used to indicate that the cartridge contains a slow acting insulin. The turtle is secured to the cartridge by a fastener 206, which may be, for example, a plastic loop that could be heated to mate or contract with the cartridge outer surface. The fastener 206 may be any suitable fastener, such as a cable, hook, screw, or adhesive, for example. Alternatively, the coding 204 may be located on the outer surface of the cartridge, either removeably or non-removeably. For example, the coding 204 may be drawn, painted, etched, or engraved onto the outer surface of the cartridge 200. Although the coding 204 is shown on the proximal end of the cartridge, it should be understood that the coding may be located at any visible area of the cartridge 200.

The visual and tactile identification coding 204 may comprise a number of different coding features, either alone or in combination. For example, the coding feature may comprise anything that represents a characteristic of the drug contained by the cartridge, such as an animal or object. The coding feature may also include a particular color. Further, the coding feature may comprise a specific texture, such as a rough or smooth surface, or a hard or flexible surface. The hard surface may be made of polycarbonate and the flexible surface may be made of PVC, for example.

Additional features which may be included as characteristics of the visual or tactile identification coding 204 are the size, shape, and orientation of the coding. For example, the coding may be large or small, and may be axial, circumferential, or radial protrusions, or ribs. Further, the coding feature may include protrusions or some other distinguishing feature to indicate differences in the drug compared to other drugs. The orientation of the coding may also be used to identify the type of drug in the cartridge. For example, the coding may project radially from the cartridge axis, in a transverse direction, or in an axial direction.

Turning now to FIG. 3, the cartridge 200 may be received by a cartridge holder 208. The cartridge holder may include a second tactile and/or visual identification coding 210. The second tactile or visual identification coding 210 may correspond to the visual or tactile identification coding 204 on the cartridge 200. For example, in this embodiment the second tactile or visual identification coding 210 is the color green to match the green turtle, indicating that a slow acting insulin is contained in the cartridge 200. Alternatively, the second tactile or visual identification coding 210 may be any other suitable coding that is related to a green turtle. For example, the surface of the label over the cartridge could be the same as the turtle. Thus, the appropriate cartridge holder 208 will be paired with the appropriate cartridge 200, and then to the appropriate drug delivery device, such as drug delivery device 100.

In operation, where the cartridge is used with a reusable drug delivery device, when a user needs to take a drug, they simply locate the tactile identification coding 204 on the cartridge 200 to determine which type of drug is contained in the cartridge. Next, the user identifies the appropriate cartridge holder 208 that matches up with the coding 204 located on the cartridge 200. The cartridge and holder may then be inserted into a drug delivery device, such as drug delivery device 100, modified to receive cartridge 200, and then be administered. Provided that the correct coding 204 is present and matched up with the correct second coding 208 on the cartridge holder, the user can be confident that they have administered the appropriate drug.

One advantage of the coded cartridge and cartridge holder system is that it may include more than one coding feature, thereby providing additional vigilance for preventing the accidental intake of the wrong type of drug by a patient.

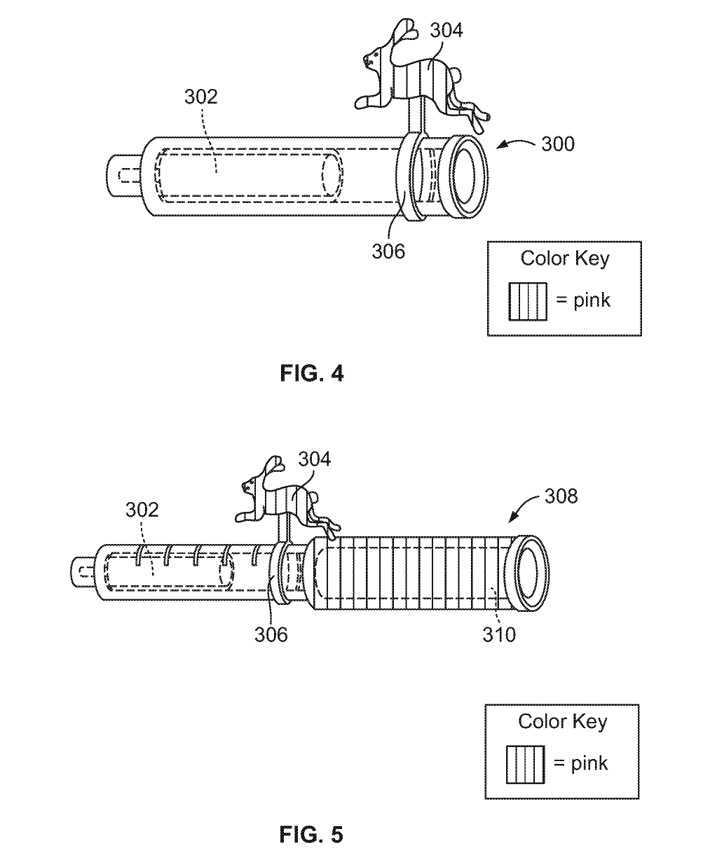

FIGS. 4 and 5 illustrate a second arrangement of a coded cartridge 300 for use with a cartridge holder, such as the cartridge holder 104 illustrated in FIG. 1a. The cartridge 300 and cartridge holder may both be used with a pen type drug delivery device, such as the drug delivery device 100 illustrated in FIG. 1a. Similar to the coded cartridge 200 illustrated in FIGS. 2-3, this cartridge configuration comprises a cartridge 300 having visual and/or tactile identification coding 304. The cartridge 300 may be inserted into a cartridge holder 308, similar to cartridge holder 104 illustrated in FIG. 1a, which may include a second tactile and/or visual identification coding 310.

The coded cartridge 300 may include a visual and tactile identification coding 304 that includes a logical symbol to visually indicate to a user the type of drug contained in the cartridge. For example, as shown in FIG. 4, the coding 304 includes a hare, which is used to indicate that the cartridge contains a fast acting insulin. The hare is secured to the cartridge by a fastener 306. Similar to the example discussed with respect to FIG. 2, the fastener may be any suitable fastener, such as a cable, hook, screw, or adhesive, for example. Alternatively, the coding 304 may be located on the outer surface of the cartridge, either removeably or non-removeably. For example, the coding 304 may be drawn, painted, etched, or engraved onto the outer surface of the cartridge 300. Although the coding 304 is shown on the proximal end of the cartridge, it should be understood that the coding may be located at any visible area of the cartridge 300.

Although shown as a single feature in FIGS. 4 and 5, it should be understood that the visual and tactile identification coding 304 may comprise a number of different features, either alone or in combination. Examples of such coding features are described above with respect to FIGS. 2 and 3.

Turning now to FIG. 5, the cartridge 300 may be received by a cartridge holder 308. The cartridge holder may include a second tactile and/or visual identification coding 310. The second tactile or visual identification coding 310 may correspond to the visual or tactile identification coding 304 on the cartridge 300. For example, in this embodiment the second tactile or visual identification coding 310 is the color pink to match the pink hare, indicating that a fast acting insulin is contained in the cartridge 300. Alternatively, the second tactile or visual identification coding 310 may be any other suitable coding that is related to a pink hare. Thus, the appropriate cartridge holder 308 will be paired with the appropriate cartridge 300, and then to the appropriate drug delivery device, such as drug delivery device 100.

In operation, where the cartridge is for use with a reusable pen type delivery device, such as the delivery device illustrated in FIG. 1a, when a user needs to take a drug, they simply locate the tactile identification coding 304 on the cartridge 300 to determine which type of drug is contained in the cartridge. Next, the user identifies the appropriate cartridge holder 308 that matches up with the coding 304 located on the cartridge 300. The cartridge and holder may then be inserted into a drug delivery device, such as drug delivery device 100, and then be administered. As long as the correct coding 304 is present and matched up with the correct second coding 308 on the cartridge holder, the user may be confident that they have administered the appropriate drug.

Although aimed primarily at the insulin market, the presently proposed coded cartridge or cartridge holder may apply to other drugs. The proposed coding system may apply to various devices, including the following examples:

a. An injector pen with a cartridge (e.g. 3 ml cylindrical glass cartridge) and a separate holder. (reusable or disposable)

b. An injector pen with a cartridge (e.g. 3 ml cylindrical glass cartridge) non-removably retained in a holder, so that the holder will be disposed of with the primary pack.

c. An injector pen where the primary pack attaches directly to the pen, e.g. an injection-moulded polymer cartridge.

d. Any drug delivery device, with any type of primary pack, e.g. inhaler, pouch.

The proposed coding system results in a number of advantages. For example, the proposed coded cartridge holder arrangements assist a user to distinguish between medicaments, thereby helping to ensure that a delivery device is used with a medicament for which the device is intended. Therefore, with the system applied to a cartridge to form a cartridge assembly, the cartridge assembly is prevented from being loaded into any other drug by loading a cartridge with an incorrect fastening mechanism. The coded cartridge or cartridge holder prevents a user from completing one or more of the following actions: fully inserting the cartridge assembly into an incorrect cartridge holder or attaching the cartridge and/or cartridge holder onto an incorrect dose setting mechanism.

The coded cartridge or cartridge holder also results in a low cost coding mechanism since the proposed cartridge holders do not require a large number of parts and can be manufactured in a cost effective manner. Moreover, there are quite a large number of different cartridge coding configurations between the cartridge, the cartridge holder, and the drug delivery device that may be used. Consequently, with proposed coding schemes, a large number of medicaments can be distinguished from one another. In addition, with the disclosed coding schemes, if a user attempts to load an incorrect cartridge assembly into a cartridge holder designed for a different cartridge assembly, the user will be alerted at an early stage of the assembly process.

In addition, the disclosed system can be used to prevent errors during manufacturing, when inserting cartridge assemblies into disposable cartridge holders or disposable devices. With an incorrect drug (and hence incorrectly coded cartridge), the user is alerted at an early stage of assembly.

Exemplary embodiments of the present disclosure have been described. Those skilled in the art will understand, however, that changes and modifications may be made to these arrangements without departing from the true scope and spirit of the present disclosure, which is defined by the claims.

1-15. (canceled)

16. 1. A tactile identification system, comprising:
a cartridge containing a drug;
an identification coding located on the cartridge, the identification coding including a symbol to visually and tactilely indicate to a user the type of drug contained in the cartridge and wherein the symbol indicates a drug action rate information, the drug action rate information including one of at least two different drug action rates.

17. The tactile identification system of claim 16, wherein the drug action rate information is provided by the outer shape of the symbol.

18. The tactile identification system of claim 16, wherein the symbol represents an animal.

19. The tactile identification system of claim 16, wherein the symbol represents one of a hare and a turtle.

20. The tactile identification system of claim 16, wherein the identification coding comprises a turtle to indicate that the cartridge contains slow acting insulin.

21. The tactile identification system of claim 16, wherein the identification coding comprises a hare to indicate that the cartridge contains fast acting insulin.

22. The tactile identification system of claim 16, further comprising a cartridge holder configured to receive the cartridge.

23. The tactile identification system of claim 22, wherein the cartridge holder includes a second tactile and/or visual identification coding that corresponds to the identification coding on the cartridge.

24. The tactile identification system of claim 23 wherein the second identification coding comprises a color.

25. The tactile identification system of claim 23, wherein the identification coding comprises a green turtle and the second identification coding comprises the color green to indicate that the cartridge contains slow acting insulin, or wherein the identification coding comprises a pink hare and the second identification coding comprises the color pink to indicate that the cartridge contains fast acting insulin.

26. The tactile identification system of claim 16, wherein the identification coding comprises only one coding feature or a plurality of coding features.

27. The tactile identification system of claim 16, wherein the symbol is secured to the cartridge by a fastener.

28. The tactile identification system of claim 16, wherein the identification coding is located on an outer surface of the cartridge.

29. A drug delivery device comprising the tactile identification system of claim 16.

30. The drug delivery device of claim 29 wherein the drug delivery device comprises a reusable drug delivery device or a disposable drug delivery device.
